(19) United States
(12) Patent Application Publication
(10) Pub. No.: US 2021/0030313 A1
(43) Pub. Date: Feb. 4, 2021

(54) HEALTH MANAGEMENT SYSTEM COMBINED WITH SMART BED SET

(71) Applicant: Wen-Chang HSIAO, Taipei City (TW)

(72) Inventor: Wen-Chang HSIAO, Taipei City (TW)

(21) Appl. No.: 16/654,108

(22) Filed: Oct. 16, 2019

(30) Foreign Application Priority Data
Jul. 30, 2019 (TW) 108126988

(51) Int. Cl. A61B 5/11; A61B 5/00; A61B 5/024; A61B 5/0205; A61G 7/05

(52) U.S. Cl. CPC A61B 5/1126; A61B 5/002; A61B 5/02405; A61B 5/4809; A61B 5/4815; A61G 7/05; A61B 5/0205

(57) ABSTRACT

Disclosed is a health management system combined with a smart bed set. Unlike conventional hospital beds used for medical treatments or medical beds used for examining patients, the system of this disclosure breaks through the limitations that patients have to go to hospitals for physical examination and reduces spaces. This system detects and accumulates physiological data during our sleeping time for a long time and analyzes the physiological data to assist an early discovery of potential diseases, and the analyzed information is provided for the personal health management of individuals or the reference for medical professionals.

### FIELD OF INVENTION

The present disclosure relates to a health management system, in particular to the health management system comprising items such as beds and pillows used in our daily life and integrated with the technologies of physiological information detection modules, databases, and analysis units to detect, compare and analyze the physiological data detected during our sleeping time, so as to discover any potential disease at an early stage and use such information as a user's health management or medical reference.

### BACKGROUND OF INVENTION

### Description of the Related Art

Many people have the habit of going to hospital for a regular physical examination, in hope of discovering any potential disease at an early stage and getting treatments timely. However, due to different reasons including personal time arrangements, the long distance to a hospital, or the inconvenient travel of the elderly, the health examination is usually held once every year, two years, or even five years. Even if the disease is found after the physical examination, the disease is often up to a certain degree of severity, and thus resulting in an economic burden of subsequent treatments of the disease, a stress of life, and/or a loss of productivity of individuals or the society and creating a great burden to the financial condition of the family or the healthcare cost of the society. Therefore, finding a way of discovering any potential disease at an early stage and taking immediate actions is an important issue that demands immediate attentions and feasible solutions.

When people go to a hospital for physical examinations, the hospital is unable to examine an early sign of sporadic abnormalities or non-obvious symptoms in a short time in addition to the limitations of time and space. For many chronic diseases with a development progression such as early arrhythmia, cardiovascular obstruction, cardiovascular sclerosis, apnea, emotional stress, epilepsy, and mental distress, the symptoms may not occur daily or frequently at the beginning, and hospitals are unable to detect or discover the disease in a single examination or within a day or two, and the patient's disease is often discovered until it reaches a certain degree of severity.

In fact, the early symptoms of many chronic diseases will show a change of physiological information first, but such change does not occur all the time or it can be detected anytime. For example, many larger hospitals have a sleep detection center for detecting and examining diseases such as sleep disorder, apnea, etc., but the users still need to go to the hospital at a specific time and stay in the hospital for a certain period of time. Furthermore, many pads connected to related instruments are attached to the patient's body in order to collect data. Besides the problem of unable of collecting the physiological data daily for a long time, the requirement of attaching detection instruments may also affect the quality of the user's sleep and the accuracy of the physiological data, and the conventional method still has to overcome the inconvenience of collecting the physiological data.

In addition, the medical systems in a hospital are unable to keep track of the examination all the time. Even if a small number of elderlies or chronic patients can afford hiring a professional nursing staff to examine and record their physiological data at home, it will take a lot of time and effort to cooperate with the professional nursing staff to attach the detection instruments and carry out the detection, recording, reading and determination, which will seriously affect the quality of life of the patients.

In view of the aforementioned drawbacks of the conventional medical systems, the discloser of this disclosure provides a health management system capable of detecting and collecting physiological data of a person unconsciously for a long time during the person's sleeping time performing related comparisons and analyses of a large quantity of data in order to discover any physiological abnormality occurred in a disease at an early stage. This health management system breaks through the limitations of the examination time and space of the hospital and achieves the effect of discovering any potential disease at an early stage without affecting the quality of life of the patient, and thus this system is definitely a good health management system.

For example, a physiological information detecting device is built in a bed set used at home daily and combined with a system capable of analyzing and determining the physiological information. The physiological information detected for a long time during the patient's sleeping time is transmitted to a mobile phone, a gateway, a computer, or a cloud platform via a wireless transmission, and such information is accumulated and the most updated detection result is compared with the previous detection information obtained at a stable period and then analyzed and determined, so that when the physiological information is obviously different from the previously accumulated physiological information or the occurring frequency of the abnormality is increased, it may be an early sign of a physical health problem which alerts people to make necessary preparations for their health management.

For example, the information such as heart rate, heart rhythm, HRV, respiration, number of body flips is detected for a period of time daily and then compared with the physiological information of a stable sleep detected for a long time or other different detection factors. Any obvious change should be noted and observed. For example, the average heart rate of normal sleep is 60, and when the average heart rate of a patient in this period of time is 73, the patient may be psychologically abnormal. When a physiological abnormality such as the frequency of HRV arrhythmia, the apnea time, or the number of their occurrence is increased, the patient should pay attention and take precautions.

In addition to these, the system may be used to accumulate the physiological information detected during sleeping time for a long time and save the accumulated physiological information in a personal database. The most updated physiological information detected daily will be compared with the physiological information stored in the database, so as to discover the initial starting time of having the symptoms of a potential disease at an early stage and put forward an early warning and achieve a good health management effect. Of course, the most updated physiological information detected daily may further be compared with a public healthcare cloud big data platform, and the system evaluates a possible health status and recommends people to go to hospital for further examination, so as to achieve the effect of assisting health management.

### SUMMARY OF THE INVENTION

Therefore, it is a primary objective of this disclosure to provide a health management system combined with a smart bed set which is different from the general health examination method used in hospitals, and to use a smart bed set with built-in devices for detecting physiological information, and a system capable of analyzing and determining the physiological information, and detecting physiological information during a patient's sleeping time unconsciously for a long time without affecting the quality of life of the patient, and the detected physiological information is transmitted to a mobile phone, a gateway, a computer, or a cloud platform via a wireless transmission, and

such information is accumulated and the most updated detection result is compared with the previous detection information obtained at a stable period and then analyzed and determined, so that when the physiological information is obviously different from the previously accumulated physiological information or the occurring frequency of the abnormality is increased, it may be an early sign of a physical health problem which alerts people to make necessary preparations for their health management. This disclosure also overcomes the difficulties of going to the hospital for a physical examination due to the factors of time and space, or the sporadic abnormality cannot be discovered simply by a single examination.

To achieve the aforementioned and other objectives, this disclosure provides a health management system combined with a smart bed set comprising: a smart bed set with a data processing module and a cable or wireless transmission module, a physiological information detection module coupled to the data processing module via a signal, a database with a physiological data statistic, an analysis unit capable of reading data from the database and coupled to the cable or wireless transmission module via a signal, and a display module coupled to the analysis unit via a signal.

Wherein, the physiological information detection module is provided for daily detecting a physiological signal of a user lying on the smart bed set during the user's sleeping time, transmitting the physiological signal to the data processing module and converting the physiological signal into physiological data, and then the physiological data are transmitted to the analysis unit through the cable or wireless transmission module, and an analysis result is generated after the analysis unit analyzes the physiological data and the physiological data statistic of the database, and the analysis result is transmitted to the database for storage, and displayed by the display module.

Compared with the prior art, the health management system of this disclosure is different from the health examination method used in the conventional medical systems, and the health management system applies the smart bed set for health management, is capable of collecting physiological data for a long time while a user is sleeping or physically and mentally situated in a stable condition, so as to improve the accuracy of the data and remind users about any potential disease through the display device after the analysis.

In addition, the design of detection during sleeping time allows users to read and determine the daily detection result obtained in an interference-free environment during sleeping unconsciously, so as to achieve the effects of discovering potential diseases at an early stage, facilitating long-term health management, and using the detection result as a medical reference. In addition, the cloud platform of the system of this disclosure may be combined with the advanced technologies of artificial intelligence (AI) and big data, so that the statistics of data can be carried out and analyzed better or the physiological data can be provided for medical research. Therefore, this disclosure not just alerts users about potential diseases in advance only, but also promotes the popularity of personal health management, and thus makes a significant contribution to the people and society.

Specifically, this disclosure has the following advantageous effects:

1. Accuracy: The accuracy of one-time examination does not represent the normal performance, and it is often a temporary or sporadic physiological change only, or may even be a physiological change that can be detected in a single examination. Comparatively, the health management system of this disclosure is capable of analyzing the detection results continuously for a long time and improving the accuracy of the detection result for a better health management.

2. Early Discovery: Diseases generally have specific development durations, and people usually go to hospital for examination when they feel sick or have serious symptoms. The health management system of this disclosure can push the discovery of diseases at an earlier time which is conducive to the healing performance and the preventive effect.

3. Hospital Barrier: The health management system of this disclosure is not limited by the time and space for going to a hospital or a medical institute for a health examination, and thus can overcome the time and space barriers of the health examination.

4. Personal Time Barrier: The health management system of this disclosure requires no medical appointment, and thus can overcome the time barrier, particularly when the user is busy.

5. Without Interference Pressure: The health management system of this disclosure can overcome the inconvenience and pressure of wearing various detection devices in a conventional medical examination and eliminate the factor of affecting the quality of normal sleep.

It is noteworthy that the health management system combined with a smart bed set in accordance with this disclosure can be used for the research of sleep disorder, apnea, and cardiovascular diseases or extended to the discovery of other diseases to offer the most direct and effective entry point. The health management system of this disclosure is expected to achieve the following four aspects in the future:

First Aspect: Automatic sleep quality detection is integrated with the health management system of this disclosure, and the mattress and environment can be adjusted and controlled via a remote end to assist users to improve their sleep quality.

Second Aspect: The health management system of this disclosure has the effect of overcoming the barrier of sleep quality and the late discovery of respiratory diseases and cardiovascular diseases.

Third Aspect: The health management system of this disclosure has the effect of discovering potential diseases at an early stage and extending to other related diseases by spectra.

Fourth Aspect: The learning techniques and algorithms of AI machines are integrated with the health management system of this disclosure to achieve the effects of predicting disease progressions, and thus the health management system of this disclosure can make contribution to the overall medical and healthcare system and personal health management and can apply the artificial intelligence (AI) to provide considerable contributions in the medical field.

### BRIEF DESCRIPTION OF THE DRAWINGS

FIG. 1 is a block diagram of a health management system in accordance with a first embodiment of this disclosure;

FIG. 2 is a block diagram of a health management system in accordance with a second embodiment of this disclosure;

FIG. 3 is a block diagram of a health management system in accordance with a third embodiment of this disclosure;

FIG. 4 is a block diagram of a health management system in accordance with a fourth embodiment of this disclosure; and

FIG. 5 is a block diagram of a health management system in accordance with a fifth embodiment of this disclosure.

### DESCRIPTION OF THE PREFERRED EMBODIMENTS

The present disclosure will become clearer in light of the following detailed description of an illustrative embodiment of this disclosure described in connection with the drawings, so that people having ordinary skill in the art can implement the present disclosure according to the description of this specification.

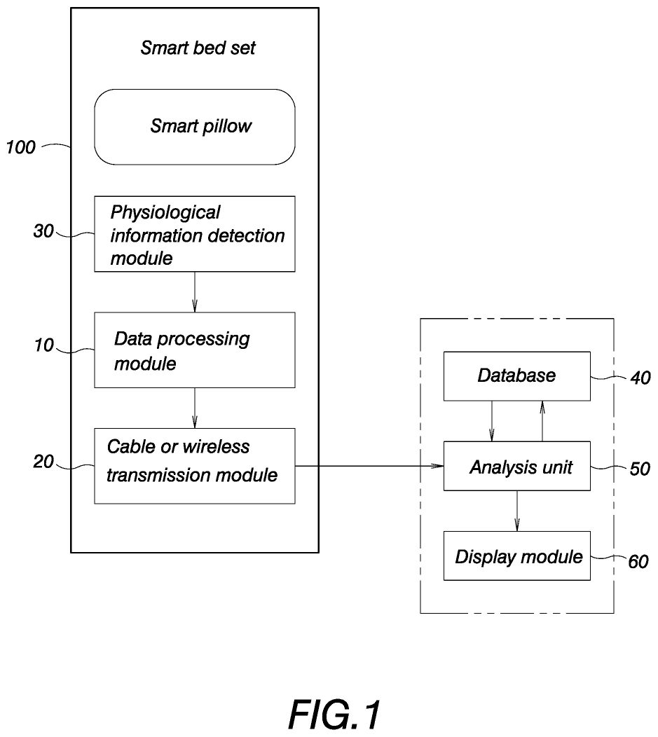

With reference to FIG. 1 for a health management system combined with a smart bed set 100 in accordance with this disclosure, the health management system is capable of collecting physiological data for a long time while a user is sleeping or physically and mentally situated in a stable condition, so as to improve the accuracy of the data. The health management system comprises a smart bed set 100 having a data processing module 10 and a cable or wireless transmission module 20, a physiological information detection module 30 coupled to the data processing module 10 via signals, a database 40 for storing the physiological data statistics, an analysis unit 50 capable of reading the physiological data statistic of the database 40 and coupled to the cable or wireless transmission module 20 via signals, and a display module 60 coupled to the analysis unit 50 via signals.

The physiological information detection module 30 is capable of detecting physiological signals of a user lying on the smart bed set 100 by a direct or indirect skin contact method. Wherein, the indirect contact method does not require an instrument attached with a detection pad in order to avoid interfering with the user's normal sleep, and the so-called physiological signals include the user's brain wave, body temperature, heart rate, pulse, blood pressure, respiration, electrocardiogram (ECG), electroencephalogram (EEG), heart rate variability (HRV), and or number of body flips.

Different from hospital beds, the aforementioned smart bed set 100 includes a smart bed and one or more smart pillows used at home. In general, people have a sleeping time of at least 4 hours, so that physiological data can be collected for a long time during the daily sleeping process, and the application of this system in the smart bed set at home is a major feature of this disclosure. The physiological information can be collected unconsciously in the long term without affecting the quality of life. Unlike the temporary or short sleeping time collection adopted in the medical institutes, this disclosure does not need to hire a professional nursing staff at home for performing daily examination and recording.

The user's physiological signal will be transmitted to the data processing module 10 and converted into physiological data, and then the physiological data will be transmitted to the analysis unit 50 via a cable or wireless transmission module 20 and the analysis unit 50 will generate an analysis result after analyzing the physiological data and the physiological data statistic stored in the database 40.

The physiological data statistic of the database 40 includes statistical data such as human brain wave, body temperature, heart rate, pulse, blood pressure, respiration, electrocardiogram (ECG), electroencephalogram (EEG), heart rate variability (HRV), number of body flips, etc., and the statistical data are standard values, wherein the analysis unit 50 analyzes and compares each standard value with each of the physiological data to form the analysis result.

For example, if the pulse of a normal adult in the statistical data is equal to 60-90 times/minute and the pulse of the user's physiological data is equal to 58 times/minute, the analysis result will indicate a too-slow pulse. If the pulse of the user's physiological data is equal to 95 times/minute, the analysis result will indicate a too-quick pulse.

The analysis result generated by the analysis unit 50 and the previous physiological data will be transmitted to the database 40 for storage, and the analysis result will be displayed on the display module 60 to inform that the user's pulse is too quick or indicate other problems, so as to achieve the effects of noticing the user for any other potential diseases, facilitating self-management or seeking for medical treatment actively, and promoting health.

Since users usually carry a smart phone, a tablet PC, or a notebook computer, or have a desktop computer or a gateway at home, and all of these devices come with various communication interfaces and transmission interfaces such as the 4G communication network, WIFI, Bluetooth, near field communication (NFC), etc., therefore the database 40 and the analysis unit 50 can be installed in form of an application (APP) and built in the smart phone, computer, or gateway to facilitate the users' remote operation, and the smart phone, computer or gateway can be used to upload the physiological data to a cloud platform for medical research. Therefore, this disclosure not just alerts users about potential diseases in advance only, but also promotes the popularity of personal health management, and thus makes a significant contribution to the people and society.

In addition, the database 40 and the analysis unit 50 may be selectively installed on the cloud platform and combined with the advanced technologies of artificial intelligence (AI) and big data, so that the physiological data can be analyzed at the cloud platform and then transmitted to the smart phone or various computers and read as described in the following embodiments.

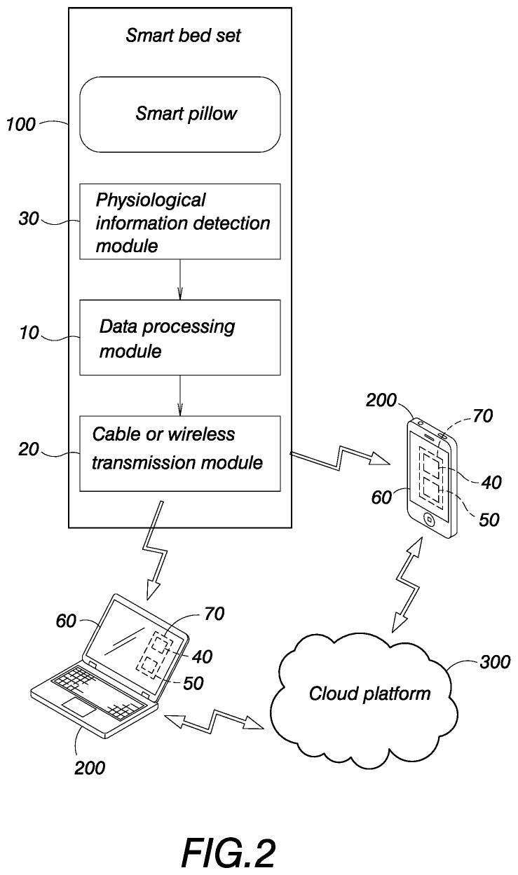

In FIG. 2, the database 40 and the analysis unit 50 are built in an application (APP) 70 of an external networking device 200, and the display module 60 is a display screen of the external networking device 200. Since the application (APP) 70 has the functions of the database 40 and the analysis unit 50, therefore the application (APP) 70 can read, store and analyze the physiological data transmitted from the cable or wireless transmission module 20 to the external networking device 200 and then display the analysis result from the display screen.

In an embodiment, the external networking device 200 is capable of downloading and updating the data of the database 40 from a cloud platform 300 by the application (APP) 70, and uploading the physiological data and the analysis result to the cloud platform 300 to obtain a better statistic and analysis of the data.

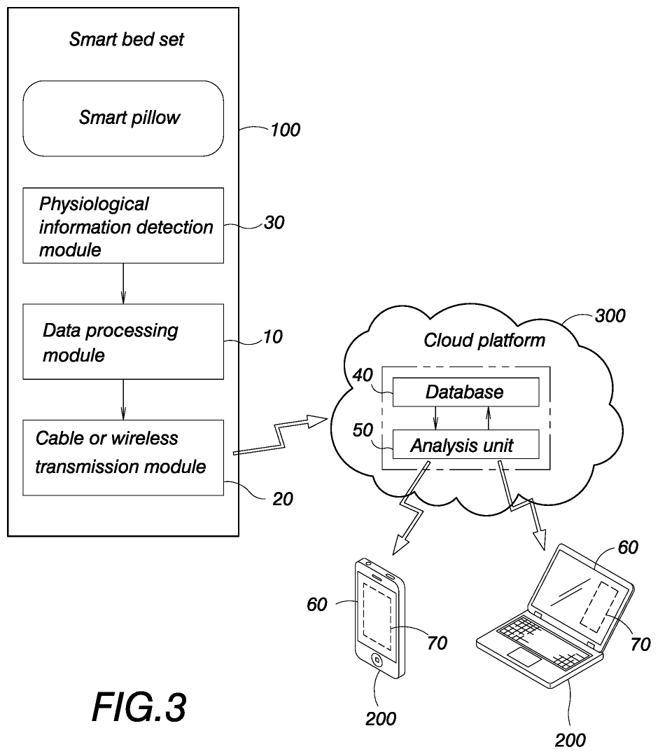

In FIG. 3, the database 40 and the analysis unit 50 are built in the cloud platform 300 and capable of updating data from the cloud platform 300. The display module 60 is a display screen of an external networking device 200, and the external networking device 200 has an application (APP) 70 compatible with the system of the database 40 and the analysis unit 50.

After the physiological data is uploaded from the cable or wireless transmission module 20 to the analysis unit 50 of the cloud platform 300 for the analysis, and then the analysis result is transmitted to the database 40 and stored into the external networking device 200, and the application (APP) 70 of the external networking device 200 can read the analysis result and display the analysis result on the display screen for the users to review.

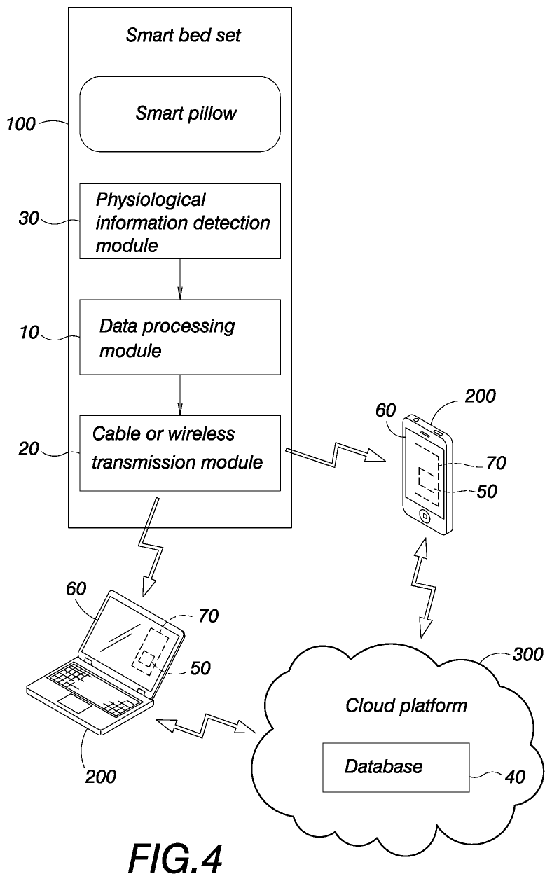

In FIG. 4, the database 40 is built in a cloud platform 300 and capable of updating the built-in data from the cloud platform 300. The analysis unit 50 is an application (APP) 70 built in an external networking device 200, and the display module 60 is a display screen of the external networking device 200, and the application (APP) 70 can download and/or read the data of the database 40 from the cloud platform 300.

After the cable or wireless transmission module 20 transmits the physiological data to the external networking device 200, and the application (APP) 70 analyzes the physiological data and the downloaded data, the analysis result is displayed on the display screen, and finally the analysis result is uploaded to the database 40 of the cloud platform 300 for its storage.

In an embodiment, the physiological information detection module 30 may be a brain wave sensor for sensing brain waves, a sound wave sensor for sensing sounds in order to detect the user's sleeping and respiration statuses, an electrocardiography (ECG or EKG) sensor for sensing the user's heart rate, an infrared ray (IR) sensor for sensing the user's body temperature, an ultra-wideband (UWB) sensor for the user's heart rate and respiration, a gyroscope for sensing the user's body movement, a photoplethysmography (PPG) sensor for sensing the user's heart rate, respiration, arterial oxygen saturation, blood pressure, and pulse, an ImageSensor for sensing the user's heart rate, a Doppler radar sensor for sensing the user's respiration and pulse, or any one of the conductive fabric, piezoresistive fabric, piezoelectric fabric, piezoelectric film, and fiber optic sensors.

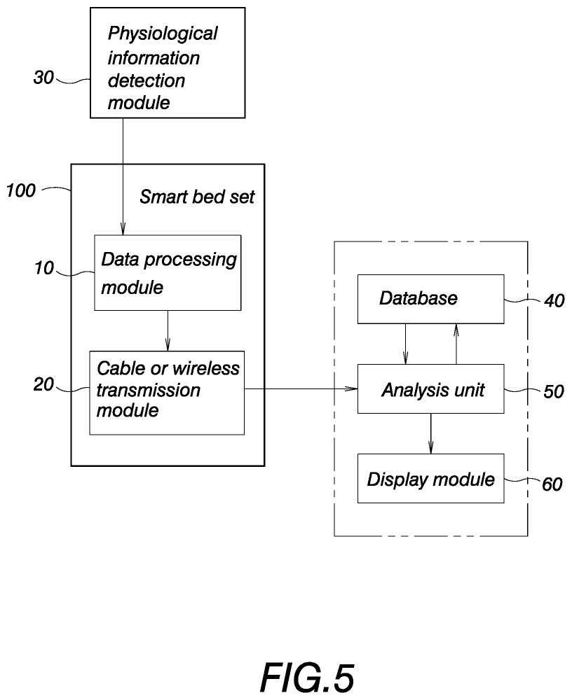

In addition, the physiological information detection module 30 may be installed onto the smart bed set 100 as shown in FIG. 1 and used for detecting physiological signals of a user lying on the smart bed set 100. The physiological information detection module 30 may be installed outside the smart bed set 100 as shown in FIG. 5. For example, the ImageSensor may be installed at the top of the smart bed set 100 and disposed at a position with a distance from the user, so that the image sensing method can be used to detect and determine the user's physiological information. The physiological information detection module 30 may also be installed in a wearable device (not shown in the figure) such as a smart bracelet or a piece of clothing which is coupled to the smart bed set 100 via signals. After the user wears the wearable device, the user's physiological signals can be detected.

Unlike the medical system, the system of this disclosure can collect the physiological data daily at home for a long term while the users are sleeping or physically and mentally situated in a stable condition, and the physiological data are analyzed and provided for the users' review and reference, so as to timely remind the users about any potential diseases, facilitate the long-term health management or use the analysis result as a medical reference. In addition, the cloud platform of the system may be combined with the technology of artificial intelligence (AI) and big data, or the analyzed physiological data may be used for medical research, promote personal health management and make a significant contribution to the people and society.

It is noteworthy that artificial intelligence (AI) will become an important milestone in the development of science and technology, and this disclosure integrates the learning techniques and algorithms of the artificial intelligence (AI) to predict disease progressions, and thus this disclosure not just can make contribution to the overall medical and healthcare system and personal health management only, but also can detect, record and analyze a large quantity of physiological data for a long time when the smart bed set becomes popular and can apply the artificial intelligence (AI) to provide considerable contributions in the medical field.

While the present disclosure has been described by means of specific embodiments, numerous modifications and variations could be made thereto by those skilled in the art without departing from the scope and spirit of this disclosure set forth in the claims.

What is claimed is:

1. A health management system combined with a smart bed set, comprising: a smart bed set with a data processing module and a cable or wireless transmission module, a physiological information detection module coupled to the data processing module via a signal, a database with a physiological data statistic, an analysis unit capable of reading data from the database and coupled to the cable or wireless transmission module via a signal, and a display module coupled to the analysis unit via a signal, wherein the physiological information detection module is provided for daily detecting a physiological signal of a user lying on the smart bed set during the user's sleeping time, transmitting the physiological signal to the data processing module and converting the physiological signal into physiological data, and then the physiological data are transmitted to the analysis unit through the cable or wireless transmission module, and an analysis result is generated after the analysis unit analyzes the physiological data and the physiological data statistic of the database, and the analysis result is transmitted to the database for storage, and displayed by the display module.

2. The health management system combined with a smart bed set as claimed in claim 1, wherein the physiological data include one selecting from the group consisting of the user's brain wave, body temperature, heart rate, pulse, blood pressure, ECG, EEG, HRV, or number of body flips; and the physiological data statistic of the database includes one selecting from the group consisting of human brain wave, body temperature, heart rate, pulse, blood pressure, respiration, ECG, EEG, HRV, and number of body flips.

3. The health management system combined with a smart bed set as claimed in claim 1, wherein the physiological information detection module is one selected from the group consisting of a brain wave sensor, a sound wave sensor, a heart rate sensor, an infrared ray (IR) sensor, an ultra-wideband (UWB) sensor, a gyroscope, a photoplethysmography (PPG) sensor, an ImageSensor, a Doppler radar sensor, a conductive fabric sensor, a piezoresistive fabric sensor, a piezoelectric fabric sensor, a piezoelectric film sensor, and a fiber optic sensors.

4. The health management system combined with a smart bed set as claimed in claim 1, wherein the database and the analysis unit are built in an application (APP) of an external networking device, and the display module is a display screen of the external networking device, and the application (APP) is provided for reading, storing and analyzing the physiological data transmitted from the cable or wireless transmission module to the external networking device, and then an analysis result is displayed from the display screen.

5. The health management system combined with a smart bed set as claimed in claim 4, wherein the external networking device is a smart phone, a computer, or a gateway capable of updating the data of the database of the application (APP) downloaded from a cloud platform and uploading the physiological data and the analysis result to the cloud platform.

6. The health management system combined with a smart bed set as claimed in claim 1, wherein the database and the analysis unit are built on a cloud platform, and the display module is a display screen of an external networking device, and the external networking device has an application (APP) compatible to the database and the analysis unit system, and the physiological data are uploaded by the cable or wireless transmission module to the analysis unit of the cloud platform and then analyzed, and the analysis result is transmitted to the database for storage and then to the external networking device, and the application (APP) is capable of reading the analysis result and displaying the analysis result by the display screen.

7. The health management system combined with a smart bed set as claimed in claim 6, wherein the database is capable of updating data from the cloud platform, and the external networking device is a smart phone, a computer, or a gateway.

8. The health management system combined with a smart bed set as claimed in claim 1, wherein the database is built on a cloud platform, and the analysis unit is built in an application (APP) of an external networking device, and the display module is a display screen of an external networking device, and the application (APP) is capable of downloading and/or reading the data of the database from the cloud platform;
the physiological data is transmitted by the cable or wireless transmission module to the external networking device and analyzed by the application (APP) and then the analysis result is displayed from the display screen and the analysis result is uploaded to the database of the cloud platform.

9. The health management system combined with a smart bed set as claimed in claim 8, wherein the database is capable of updating the built-in data from the cloud platform, and the external networking device is a smart phone, a computer, or a gateway.

10. The health management system combined with a smart bed set as claimed in claim 1, wherein the physiological information detection module is installed on the smart bed set, or outside the smart bed set, or a wearable device worn by the user, and the wearable device is a smart bracelet or a piece of clothing.
